(19) United States
(12) Patent Application Publication
(10) Pub. No.: US 2008/0096206 A1
(43) Pub. Date: Apr. 24, 2008

(54) Polynucleotide Sequencing Using a Helicase

(76) Inventor: Daniel Henry Densham, Devon (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/832,441

(22) Filed: Aug. 1, 2007

Related U.S. Application Data
(63) Continuation of application No. 09/937,784, filed on Jan. 28, 2002, now abandoned.

(30) Foreign Application Priority Data
Apr. 6, 1999 (GB) 9907812.3

(51) Int. Cl. C12Q 1/68; C12M 1/00

(52) U.S. Cl. 435/6; 435/287.2

(57) ABSTRACT

The present invention pertains to a method for sequencing a polynucleotide, comprising the steps of: (i) reacting a target polynucleotide with a helicase/primase enzyme (which may be immobilised), under conditions suitable for enzyme activity; and (ii) detecting the interaction between the enzyme and a nucleotide on the target, by measuring radiation.

### FIELD OF THE INVENTION

This invention relates to a method for determining the sequence of a polynucleotide.

### BACKGROUND OF THE INVENTION

There is considerable interest in sequencing polynucleotides. A brief summary, and description of an effective method, will be found in WO-A-99/05315.

### SUMMARY OF THE INVENTION

The present invention is based on the realisation that the measurement of electromagnetic radiation can be used to detect a conformational and/or mass change in a helicase and/or primase which occurs when these proteins unwind double-stranded DNA (dsDNA) into single-stranded (ssDNA), using energy from NTP hydrolysis.

According to the present invention, a method for sequencing a polynucleotide comprises the steps of:
(i) reacting a target polynucleotide with a helicase/primase enzyme, and the source of NTP, under conditions suitable for helicase activity (i.e. DNA unwinding utilising the energy from NTP hydrolysis); and
(ii) detecting the separation and/or proximity of a specific base or base pair via the action of the helicase, by measuring radiation.

Using a helicase in order to determine the sequence of a polynucleotide offers several advantages for the success of this method. Firstly, the problem of secondary structures that exist within polynucleotide molecules is reduced since helicases encounter and overcome these structures within their natural environment. Secondly, helicases offer the ability to directly sequence double-stranded DNA at room temperature. This ability offers advantages in terms of ease of manipulation of target polynucleotides and the possibility of sequencing long polynucleotide templates.

The radiation may be applied to a sample using a number of techniques, including surface-sensitive detection techniques (in which instance the helicase enzyme will be bound to a solid support), where a change in optical response at a solid optical surface is used to indicate a binding interaction at the surface. In a preferred embodiment of the invention, the technique used is evanescent wave spectroscopy, in particular surface plasmon resonance (SPR) spectroscopy.

### BRIEF DESCRIPTION OF THE DRAWINGS

FIG. 1 shows the results from the sequencing experiment, as a plot of response (RU) versus time (T; sec). This shows detection of each nucleotide being incorporated into the nascent chain. The results show a sequence complementary to that of the target polynucleotide.

### DESCRIPTION OF THE INVENTION

In an embodiment of the invention, the energy available to the helicase, in the form of NTP, is under strict control. That is, the motion of the helicase along the DNA strand to be sequenced is regulated via direct control of the concentration of an energy source molecule in the region of its binding site and hence availability to the helicase molecule. This allows enzyme activity to be regulated so as to promote the action of measuring radiation in order to identify a base or base pair within proximity to the helicase or helicase complex.

Alternatively, the control of DNA unwinding, and hence sequencing progress, may be accomplished by controlling the ability of the helicase enzyme to undergo a conformational change that allows it to either carry out hydrolysis and/or move along a polynucleotide. This may be achieved by engineering (via state-of-the art genetic manipulation techniques) a helicase (or molecule associated with it) such that it contained a chemical/moiety group or groups that enable the molecule to convert or transduce radiation into a conformational change. The selective control of helicase activity is carried out in a way that ensures the detection of each nucleotide. The method may therefore proceed on a real-time basis, to achieve a high rate of sequence analysis. A preferred method of control is described in the copending PCT Application in the same name and filed on the same day, the contents of which are incorporated herein by reference.

The present method for sequencing a polynucleotide involves the analysis of the conformational/kinetic interaction between a helicase enzyme and a target polynucleotide. Measurement of conformational/kinetic interaction is carried out by monitoring the changes in or absorption of electromagnetic or other radiation that occurs if the reaction proceeds.

The term “polynucleotide” is used herein as to be interpreted broadly, and includes DNA and RNA, including modified DNA and RNA, as well as other hybridising nucleic acid-like molecules, e.g. peptide nucleic acid (PNA).

The term “helicase” is used herein as to be interpreted broadly, and pertains to ubiquitous proteins that unwind double-stranded polynucleotides into single-stranded polynucleotides, and may or may not utilise energy from NTP hydrolysis to achieve this (Dean et al, J. Biol. Chem. (1992) 267:14129-14137; Bramhill et al, Cell (1988) 54:915-918; Schions et al, Cell (1988) 52:385-395).

The first helicase was discovered and classified more than 20 years ago (Abdel-Monem et al, Eur. J. Biochem. (1976) 65: 411-449 & 65:431-440). New helicases are continually being discovered and characterised from prokaryotic, eukaryotic and viral systems. All these molecular systems are within the scope of the invention.

The helicase used in the invention may be of any known type. For example, the helicase may be any DNA-dependant DNA helicase, e.g. E. coli DnaB Helicase (Xiong et al, J. Mol. Biol. (1996) 259: 7-14.). If the target polynucleotide is a RNA molecule, then the helicase may be a RNA-dependent helicase or a helicase that is able to act on both forms of polynucleotide. A digestion enzyme, e.g. an exonuclease, or a topoisomerase, may also be used.

In a preferred embodiment of the invention, the helicase is bacteriophage T7 gp4 helicase (Egelman et al, Proc. Natl. Acad. Sci. USA, (1995) 92:3869-3873). In a further preferred embodiment of the invention, the helicase is either E. coli RuvB helicase (Stasiak et al, Proc. Natl. Acad. Sci. USA, (1994) 91:7618-7622), E. coli DnaB Helicase (Xiong et al, J. Mol. Biol. (1996) 259: 7-14), or simian virus 40 large T helicase (Dean et al, J. Biol. Chem. (1992) 267:14129-14137).

A large number of helicases characterised to date have either been shown to be oligomeric in their active form, or this is assumed to be the case.

At present, helicases have been classified into families according to primary structure (Gorbalenya et al, Current Opin. Struct. Biol. (1993) 3:419-429) but can also be grouped on the basis of oligomeric state or polarity of polynucleotide unwinding (Lohman et al, Annu. Rev. Biochem (1996) 65:169-214 & Bird et al, Current. Opin. Struct. Biol (1998) 8:14-18). A large number of putative helicases have been identified through sequence homology in prokaryotes, eukaryotes and viruses (Gorbalenya et al, Current Opin. Struc. Biol. (1993) 3:419-429). Although many helicases appear to function as either hexamers or dimers (Lohman et al, Annu. Rev. Biochem (1996) 65:169-214), some are monomeric, such as the PcrA helicase (Bird et al, Nucleic Acids Res. (1998) 26:2686-2693) and the NS3 helicase (Porter et al, J. Biol. Chem. (1998) 273:18906-18914) for example. Other helicases, such as Rep helicase, may also exist in monomeric form (Bird et al, Nucleic Acids Res. (1998) 26:2686-2693).

In a preferred embodiment of the invention, PcrA helicase from the moderate thermophile Bacillus stearothermophilus is utilised in order to take advantage of the manipulative stability of a monomeric system. PcrA helicase has been shown to be an essential enzyme in Bacillus subtilis (Petit et al, Mol. Microbiol. (1998) 29:261-274) and Staphylococcus aureus (Lordanescu et al, Mol. Gen. Genet. (1993) 241:185-192) involved in repair and rolling cycle replication (Petit et al, Mol. Microbiol. (1998) 29:261-274 & Soultanas et al, Nucleic Acids Res. (1999) 256:350-355). PcrA also shows considerable homology to both E. coli UvrD and Rep.

Typically, the method is carried out by applying electromagnetic radiation, by using techniques of surface plasmon resonance or nuclear magnetic resonance. However, other techniques which measure changes in radiation may be considered, for example spectroscopy by total internal reflectance fluorescence (TIRF), attenuated total reflection (ATR), frustrated total reflection (FTR), Brewster angle reflectometry, scattered total internal reflection (STIR) or evanescent wave ellipsometry.

Techniques other than those requiring electromagnetic radiation are also envisaged, in particular photochemical techniques such as chemiluminescence, and gravimetric techniques including resonant systems such as surface acoustic wave (SAW) techniques and quartz crystal microbalance (QCM) techniques.

Surface plasmon resonance (SPR) spectroscopy is a preferred method, and measures the properties of a solution by detecting the differences in refractive index between the bulk phase of the solution and the evanescent wave region. Incident monochromatic light is reflected at a specific angle of a solid optical (sensor chip) surface on the opposite side to the sample under study. The light extends into the sample for a short distance and is affected by an interaction at the surface.

Suitable sensor chips are known in the art. Typically, they comprise an optically transparent material, e.g. glass, and a thin reflective film, e.g. silver or gold. For a review of SPR spectroscopy, see EP-A-0648328.

Nuclear magnetic resonance (NMR) spectroscopy is another preferred method, and measures the magnetic properties of compounds. Nuclei of compounds are energetically orientated by a combination of applied magnetic field and radio-frequency radiation. When the energy exerted on a nucleus equals the energy difference between spin states (the difference between orientation parallel or anti-parallel to the direction of the applied fields), a condition known as resonance is achieved. The absorption and subsequent emission of energy associated with the change from one spin state to the other are typically detected by a radio-frequency receiver.

An important aspect, although not essential, of the present invention is the use of a helicase enzyme/complex immobilised onto a solid support. Immobilisation of the helicase offers several important advantages for the success of this method. Firstly, the problem of random “noise” associated with measuring energy absorption in soluble molecules is reduced considerably. Secondly, the problem of noise from the interaction of any substrate (e.g. NTP sources) not directly involved with the helicase is reduced, as the helicase can be maintained within a specifically defined area relative to the field of measurement. This is particularly relevant if the technique used to measure the changes in radiation requires the measurement of fluorescence, as in TIRF, where background fluorescence increases as the nascent chain grows. Also, if SPR spectroscopy is used, the helicase reactions are maintained within the evanescent wave field and so accurate measurements can be made irrespective of the size of the polynucleotide. Finally, as neither the target polynucleotide nor the oligonucleotide primer is irreversibly attached to the solid surface, it is relatively simple to regenerate the surface, to allow further sequencing reactions to take place using the same immobilised helicase or helicase complex.

Immobilisation may be carried out using standard procedures known in the art. In particular, immobilisation using standard amine coupling procedures may be used, with attachment of ligand-associated amines to, say, a dextran or N-hydroxysuccinimide ester-activated surface. In a preferred embodiment of the invention, the helicase is immobilised onto a SPR sensor chip surface where changes in the refractive index may be measured. Examples of procedures used to immobilise biomolecules to optical sensors are disclosed in EP-A-0589867, and Löfas et al., Biosens. Bioelectron. (1995) 10: 813-822.

In yet another embodiment of the invention, the DNA molecule could be attached to a bead. For example, one end may be biotinylated and attached to a streptavidin-coated polystyrene sphere (Chu et al, Optical Society of America, Washington, D.C., (1990), 8:202) and held within an optical trap (Ashkin et al, Opt. Lett. (1986) 11:288) within a flow cell. As the helicase (under external control) makes its way along the polynucleotide being sequenced, the polynucleotide can be moved in space via the optical trap (also known as optical tweezers) and hence keep the helicase within the field of detection. This system may also work in reverse, the bound helicase being held by the optical trap.

A further preferred embodiment of the present invention is the use/detection of single enzyme(s)/enzyme systems such that conformational changes can be monitored with or with labels. Use of, for example, a single labelled polymerase offers several important advantages for the success of this method/embodiment. Firstly, the problem of intermittent processivity of non-polymerase molecules (e.g. exonucleases) in single polynucleotide fragment environments is reduced considerably. Secondly, the problem of having to detect single labelled molecules (i.e. nucleotides) within a flow stream and its inherent noise problems is avoided. This also removes the problem of uncontrolled nucleotide binding to surfaces related to or within the template polynucleotide. The use of any number of techniques known in the art for determining/monitoring single molecule conformational dynamics, molecular interactions, enzymatic activity, reaction kinetics, molecular freedom of motion, alterations in activity and in chemical electrostatic environment, are considered to be within the scope of the present invention. Such techniques include, but are not limited to, Fluorescence energy transfer (FRET) (Ha et al., (1996) Proc. Natl. Acad. Sci. USA 96:893), Fluorescence Lifetime Microscopy (FLIM), single molecule polarisation/anisotropy measurements and Atomic Force Microscopy (AFM) measurements.

The following Example illustrates the invention.

### EXAMPLE

The following analysis was carried out on a modified BIAcore® 2000 system (BIAcore AB, Uppsala, Sweden) with a sensor chip CM5 (Research grade, BIAcore AB) as the optical sensor surface. The instrument was provided with an integrated m-fluidic cartridge (IFC) which allows analysis in four cells by a single sample-injection.

Preparation of PcrA Helicase

PcrA helicase was prepared according to Bird et al, Nucleic Acids Res. (1998) 26:2686-2693, using hydrophobic interaction chromatography on heparin-Sepharose, to purify the helicase at low salt concentrations. Trace protein contaminants were removed by gel filtration. PcrA concentration was determined spectrophotometrically using a calculated extinction coefficient of 0.76 OD mg⁻¹ mL⁻¹ 1 cm⁻¹ at 280 nm as described by Dillingham et al, Biochemistry (2000) 39:205-212.

Immobilisation of the Helicase

Immobilisation of the helicase to the sensor chip was carried out according to Jönsson et al., Biotechniques (1991); 11:620-627). Briefly, the sensor chip environment was equilibrated with Hepes buffer (10 mM Hepes, 150 mM NaCl, 0.05% surfactant P20 (BIAcore AB, Uppsala, Sweden), pH 7.4). Equal volumes of N-hydroxysuccinimide (0.1 M in water) and N-ethyl-N′-(dimethylaminopropyl)carbodiimide (EDC) (0.1 M in water) were mixed together and injected across the chip (CM5) surface, to activate the carboxymethylated dextran. The PcrA helicase (160 μl) was mixed with 10 mM sodium acetate (100 μl, pH 5) and injected across the activated surface. Finally, residual N-hydroxysuccinimide esters on the sensor chip surface were reacted with ethanolamine (35 μl, 1 M in water, pH 8.5), and non-bound helicase was washed from the surface. The immobilisation procedure was performed with a continuous flow of Hepes buffer (5 μl/min) at a temperature of 25° C.

Oligonucleotides

The target and primer oligonucleotides defined as SEQ ID No.1 and SEQ ID No.2 were used. The two polynucleotides were reacted under hybridising conditions to form the target-primer complex.

The primed DNA was then suspended in buffer (20 mM Tris-Hcl, pH 7.5, 8 mM MgCl₂, 4% (v/v) glycerol, 5 mM dithiothreitol (DDT), 40 mg bovine serum albumin) containing 0.5 mM 1-(nitrophenyl)ethyl-caged ATP (caged at the 5′ position). This NPE-caged ATP is a non-hydrolysable and photoactivated analogue of ATP.

The primed DNA and NPE-caged substrate solution was then injected over the PcrA helicase on the sensor chip surface at a flow rate of 5 μl/min, and allowed to bind to the helicase via the formation of a PcrA/DNA/NPE-ATP complex.

In order to prevent template dissociation from the helicase/chip surface, a continues flow of Hepes buffer containing 0.5 mM ADP was maintained over the chip surface.

DNA Sequencing

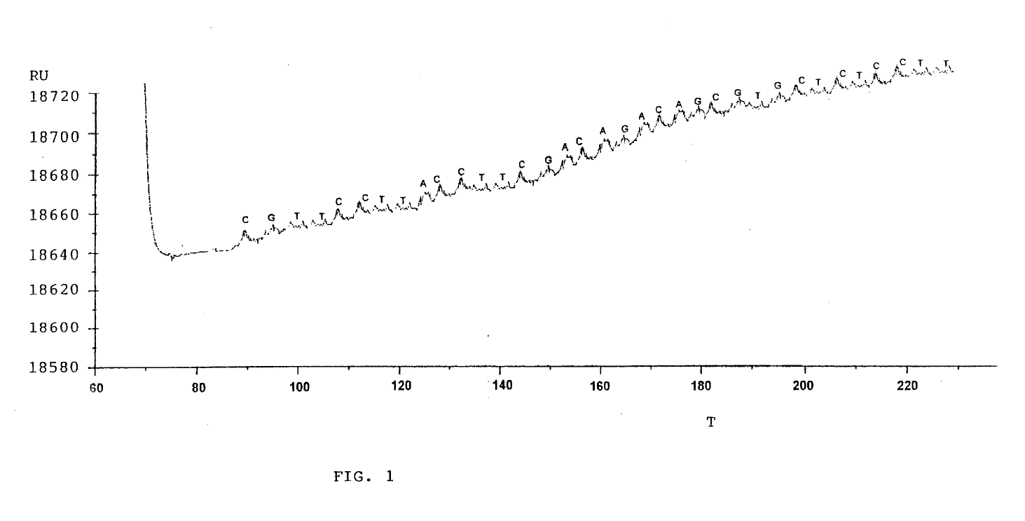

DNA sequencing was conducted by the method described in WO-A-99/05315, using the apparatus shown there in FIG. 1 of WO 99/05315, but using only one focusing assembly (5) for pulsing monochromatic light into the cell.

At the start of the experiment, a flow of Hepes buffer containing 0.5 mM is maintained across the chip surface at a flow rate of 30 μl/min and at a temperature of 25° C., and a data collection is recorded at a rate of 10 Hz. Monochromatic light at a wavelength of 260 nm is pulsed via the focusing assembly (5) to remove the blocking group on the ATP molecule within the helicase reaction site. This allows the helicase to hydrolyse the ATP to ADP, utilising the energy released to move one base pair further allow the polynucleotide. The conformational change associated with the base movement is then detected by the p-polarised light of the SPR device which is wavelength-modulated in order to produce an SPR spectrum. No further movement/unwinding occurs, since there is no ATP substrate available to the helicase to hydrolyse as an energy source.

Hepes buffer containing 0.5 mM NPE-caged ATP is then transiently introduced into the fluidic cell (6) at a flow rate of 30 μl/min and a temperature of 25° C. This allows a new ATP-substrate complex to be formed within the immobilised helicase on the chip surface. Subsequently, Hepes buffer containing 0.5 mM ADP is again introduced into the flow cell and again the complex bound ATP is uncaged and the substrate dsDNA is again unwound by a single base pair and its identity determined.

The accompanying drawing shows the results from the sequencing experiment, as a plot of response (RU) versus time (T; sec). This shows detection of each nucleotide being incorporated into the nascent chain. The results show a sequence complementary to that of the target polynucleotide.

1. A method for sequencing a polynucleotide, comprising the steps of:
(i) reacting a target polynucleotide with a helicase enzyme or a primase enzyme, under conditions suitable for enzyme activity; and
(ii) detecting the interaction between the enzyme and a nucleotide on the target polynucleotide, by measuring radiation.

2. The method, according to claim 1, wherein the radiation is electromagnetic.

3. The method, according to claim 1, wherein step (ii) comprises using surface plasmon resonance.

4. The method according to claim 1, wherein step (ii) comprises using nuclear magnetic resonance.

5. The method, according to claim 2, wherein step (ii) comprises using surface plasmon resonance.

6. The method, according to claim 2, wherein step (ii) comprises using nuclear magnetic resonance.

7. The method according to claim 1, wherein the enzyme is immobilised on a solid support.

8. A method for sequencing a polynucleotide, comprising the steps of:
(i) reacting a target polynucleotide with a helicase enzyme and a primase enzyme under conditions suitable for enzyme activity; and
(ii) detecting the interaction between the enzymes and a nucleotide on the target polynucleotide, by measuring radiation.

9. The method, according to claim 8, wherein the radiation is electromagnetic.

10. The method, according to claim 8, wherein step (ii) comprises using surface plasmon resonance.

11. The method according to claim 8, wherein step (ii) comprises using nuclear magnetic resonance.

12. The method, according to claim 9, wherein step (ii) comprises using surface plasmon resonance.

13. The method, according to claim 9, wherein step (ii) comprises using nuclear magnetic resonance.

14. The method according to claim 8, wherein the enzymes are immobilised on a soild support.

15. A sensor chip comprising a helicase enzyme, a primase enzyme, or both a helicase enzyme and a primase enzyme immobilised thereon.


SEQUENCE LISTING (2 sequences)

SEQ ID NO: 1 - DNA, 53 bp, Artificial Sequence
  Synthetic oligonucleotide used as target polynucleotide
caaggagaggacgctgtctgtcgaaggtaaggaacggacgagagaagggagag

SEQ ID NO: 2 - DNA, 17 bp, Artificial Sequence
  Synthetic oligonucleotide used as primer
ctctcccttctctcgtc